(19) United States
(12) Patent Application Publication
(10) Pub. No.: US 2008/0175803 A1
(43) Pub. Date: Jul. 24, 2008

(54) Cosmetic Compositions, Methods of Applying Said Compositions, and Methods of Making Said Compositions

(76) Inventor: Steven N. Gordon, New York, NY (US)

Correspondence Address: NIXON & VANDERHYE, PC, 901 NORTH GLEBE ROAD, 11TH FLOOR, ARLINGTON, VA, 22203, US

(21) Appl. No.: 11/848,590

(22) Filed: Aug. 31, 2007

Related U.S. Application Data
(63) Continuation of application No. PCT/US2006/007592, filed on Mar. 3, 2006.
(60) Provisional application No. 60/658,180, filed on Mar. 4, 2005.

(51) Int. Cl. A61K 8/19; A61Q 1/02; A61Q 1/06; A61Q 1/10; A61Q 1/12; A61Q 17/04

(52) U.S. Cl. 424/59; 424/63; 424/64

(57) ABSTRACT

A cosmetic composition includes talc or a talc substitute, mica, and iron oxide. The cosmetic composition optionally includes starch, tapioca, and zinc oxide. When applying the cosmetic composition to a user's skin as a body powder or the like, the composition enables the user to control the powder tint.

### CROSS-REFERENCES TO RELATED APPLICATIONS

This application is a continuation of PCT International Patent Application No. PCT/US2006/007592 filed Mar. 3, 2006, pending, which claims the benefit of U.S. Provisional Application Ser. No. 60/658,180, filed Mar. 4, 2005, the entire content of each of which is hereby incorporated by reference.

### BACKGROUND AND SUMMARY OF THE INVENTION

The invention is generally related to a cosmetic composition for human use, in varying shades, that may complement and blend in with different skin tones. The invention is also generally related to a process of applying the cosmetic composition to a user's skin. The invention is also generally related to a process of manufacturing or mixing the cosmetic composition.

In light of the increased purchasing power of African Americans, Hispanics, Asians and other ethnic groups both in the United States and internationally, there is a need and compelling demand for skin-care products which can address this group's specific and unique requirements—as a function of their skin tones.

Although African Americans, Hispanics, Asians and other ethnic groups use body powder, many find that there is no acceptable body powder on the market today designed to blend in with naturally darker skin tones. Instead, people of color typically have to resort to using baby powder, which leaves a white, flaky residue that is often embarrassing to the user.

People of color often use disproportionate amounts of talcum powder because of extra skin sebaceousness, cultural perceptions, to prevent oil build-up on the skin, to minimize body odor and for a refreshing feeling.

People of color have very specific skin care needs due to several reasons, including, ashiness—the top layers of skin cells are constantly drying and flaking off, because skin is in a constant state of renewal, with new cells steadily working upward from subdermal layers to the epidermis; an abundance of dead cells—notably when skin is dry or aging—shows up as a white or grey ashiness on the surface; keloids—darker skin has a tendency for keloids to be darker than surrounding tissue; melanin imbalances—darker skin is chemically distinguished from other types of skin by its high melanin content (melanin is a natural pigment, occurring only in the topmost layers of the skin. Because of its uneven distribution in darker skin, it causes lighter and darker patches which may be very troubling cosmetically); oiliness—surface oil shows up quite clearly on dark skin, therefore excessive oil is an annoying problem for people of color and vitiligo—the loss of pigment in areas of skin that may be of any shape or size (the borders of such areas may be darker than the person's normal skin tone).

Common usage of conventional baby powders may leave a white residue on the consumer's body which is embarrassing and cosmetically unpleasant.

Conventional body/baby powder is generally placed on or about the body because of oiliness issues, to mask body odor and to provide a refreshing feeling.

The present invention is unique in that it is believed that no body powder with similar attributes exists that is designed specifically for people with darker skin tones. Conventional body powders, due to their white color, may leave a flaky, crusty, white residue on all body areas to which it is applied. A cosmetic composition according to the present invention not only blends with the users' skin but complements his/her color and masks any skin imperfections while outperforming conventional powders' absorbency. It is believed that there are currently no comparable products in the market place that provides these benefits.

In its bottled state, a powder according to one aspect of the present invention may appear whitish, beigeish, or brownish in color. In general, the user selects, as a function of his/her skin color, one of a few different tones, which upon application may blend into the natural coloration of the skin.

When applied to a user's skin, a cosmetically desired tint or shade (e.g., darkness) of the powder may be obtained in direct proportion to the amount of friction applied to the powder upon the skin via a rubbing or massaging motion. Although not wishing to be bound by this theory, increased rubbing of the powder and/or friction on the skin may, for example, cause breakage of pigments in the powder composition. If a user desires less shading (e.g., a lighter shade), she would massage it onto her skin lightly, then allow the excess pigment to be removed by, for example, being brushed away or rolling off. Conversely, if the user desires more shading, she would rub the powder into her skin more vigorously, thus achieving the desired darkness.

The powder, furthermore, may have a chameleon effect. Depending on the user's skin tone, it may appear as having different shades. When applied to a user with a darker skin tone, the powder may appear darker. When applied to a user with a lighter skin tone, the powder may appear lighter, even though the amount of rubbing is similar in both instances.

The powder, moreover, may contain at least one ingredient that aids in protecting the skin from UV rays. Accordingly, the composition may assist in maintaining healthy skin.

Chemical sunscreen ingredients may be designed to absorb portions of the UVB (e.g., burning rays) or UVA (e.g., may cause of aging and skin cancer) rays and are often used in combination to achieve adequate UVB and at best, limited UVA protection. Zinc oxide is a clear sunscreen ingredient available today that may protect against the full “broad spectrum” of sun rays. Additionally, unlike most chemical sunscreen ingredients, zinc oxide may be stable and may not degrade in the sun.

The percentage of zinc oxide may be proportional to its SPF rating meaning, for example, 15% by quantity is equal to an SPF of 15 effectively blocking 93% of the sun's burning rays. Typically, however, body powders rarely contain more than 1-3%.

Additional usages for the powder may involve different marketing strategies, with the same basic formula, for people of lighter skin tones, so that they may enjoy its application through usage as a sunless tanner. The light skinned user could apply the product, in the same manner, with the goal of obtaining temporary darker skin (akin to a suntan) without any ill effects of damaging sun exposure.

Another use of the product may involve a shimmer product—in substantially the same formula but with shimmer for cosmetic purposes.

### DESCRIPTION OF THE PREFERRED EMBODIMENTS

In general, the composition of the present invention may comprise, for example, at least three components: at least one talc (or talc substitute); at least one mica; and at least one iron oxide. It is believed that iron oxide provides the primary source for the properties of this composition.

Unless specifically stated otherwise, all percentages are by weight of total composition, and all percentages and other numeric amounts are approximate.

As used herein, “talc” (or, alternatively, “talcum”) refers not only to powdered hydrous magnesium silicate but also to other chemicals and/or minerals routinely found in commercially available talc-containing compositions. Suitable examples for use in the composition of the present invention include, without limitation, aluminum magnesium silicate. Moreover, talc may comprise aluminum, iron, manganese, and/or titanium. Furthermore, “talc” as used herein does not refer to any specific size of particle or any specific particle size distribution, so long as the composition is cosmetically acceptable. A person of ordinary skill in the art will understand that suitable, non-limiting examples of talc substitutes may include, for example: starch (including, for example, corn starch, rice starch, potato starch, sago starch, tapioca starch, wheat starch, modified starch, etc., and mixtures thereof); calcium carbonate; silica (including fumed/precipitated silica); kaolin; bentonite; clay; calomine; and the like. A skilled artisan will appreciate that this list is not intended to be exhaustive and that talc may be partially or entirely substituted with at least one component having substantially similar properties to talc when used in a cosmetic composition (i.e., the talc substitute must be a commercially acceptable alternative to talc from the viewpoint of a reasonable consumer). In this regard, the talc or talc substitute should act, for example, as an absorbent and/or pigment carrier. Suitable talcs include, for example: Luzenac Imperial 200 sold by Luzenac, Inc.

Cosmetic compositions of the present invention preferably comprise, for example, 10-90% talc and/or talc substitute, and all ranges therebetween; more preferably, 25-90% talc and/or talc substitute, and all subranges therebetween; even more preferably, 40-90% talc and/or talc substitute, and all subranges therebetween; even more preferably, 50-90% talc and/or talc substitute, and all subranges therebetween; even more preferably, 60-80% talc and/or talc substitute, and all subranges therebetween; and most preferably 70% talc and/or talc substitute.

As used herein, “mica” broadly refers to all types of mica, including, for example, sericite, biotite, muscovite, lepidolite, phlogopite, lithia mica, vermiculite, illite, paragonite, synthetic mica, and the like. Of these, sericite is preferred. Cosmetic compositions of the present invention preferably comprise, for example, 1-50% mica, and all subranges therebetween; more preferably, 5-40% mica, and all subranges therebetween; even more preferably, 5-20% mica, and all subranges therebetween; even more preferably, 10-20% mica, and all subranges therebetween; even more preferably, 13-17% mica, and all subranges therebetween; and most preferably, 15% mica. As the percentage of mica in the cosmetic composition increases, the percentage of talc (or talc substitute) typically decreases. For example, if the composition comprises 60% talc, the composition may comprise 25% mica. For another example, if the composition comprises 70-75% talc, the composition may comprise 15% mica. Suitable micas include, for example: “Sericite BC” sold by Mineral and Pigment Solutions, Inc. (MPSI).

As used herein, “iron oxide” broadly refers to all types of iron oxides, including (but not limited to), for example, red iron oxide, yellow iron oxide, brown iron oxide, and black iron oxide. Of these, brown iron oxide and yellow iron oxide are preferred. Cosmetic compositions of the present invention preferably comprise, for example, 1-10% iron oxide, and all subranges therebetween; more preferably, 3-7% iron oxide, and all subranges therebetween; and most preferably, 4-5% iron oxide, and all ranges therebetween. Suitable iron oxides include, for example: Light Brown Cosmetic Iron Oxide CG-925; Brown Iron Oxide GC-940; Brown Iron Oxide GC-994; Brown Iron Oxide GC-929; Brown Iron Oxide GC-974; Yellow Iron Oxide YO-2087; and Yellow Iron Oxide YO-3587, all of which are sold by MPSI.

The cosmetic composition may comprise, for example, at least one fragrance. As used herein, “fragrance” broadly refers to any substance (including, for example, powders and/or liquid) that provides a scent to the cosmetic composition. This scent may, for example, be similar to the scent of baby powder. Cosmetic compositions of the present invention preferably comprise, for example, 0.001-10% fragrance, and all subranges therebetween; more preferably, 0.1%-7.5% fragrance, and all subranges therebetween; even more preferably, 4-6% fragrance, and all subranges therebetween; and most preferably, 3-5% fragrance, and all subranges therebetween. Suitable fragrances include, for example: “Soothing Vanilla,” “Lavender Blossoms,” and “Chamomile Bouquet” sold by Carrubba, Inc.

The cosmetic composition may comprise, for example, at least one starch selected from the group consisting of corn starch, rice starch, potato starch, sago starch, tapioca starch, wheat starch, modified starch, and the like. Of these, corn starch is preferred. Cosmetic compositions of the present invention preferably comprise, for example, 0-90% starch, and all subranges therebetween; more preferably, 1-70% starch, and all subranges therebetween; even more preferably, 5-60% starch, and all subranges therebetween; even more preferably, 5-30% starch, and all subranges therebetween; even more preferably, 5-20% starch, and all subranges therebetween; and most preferably, 10-15% starch, and all subranges therebetween. As noted above, starch can be a talc substitute, and as the percentage of talc in the cosmetic composition decreases, the percentage of starch may increase. For example, if the composition comprises 10-30% talc, the composition may comprise 30-60% starch. For another example, if the composition comprises 60-80% talc, the composition may comprise 5-20% starch. For another example, if the composition comprises 70-75% talc, the composition may comprise 5-10% starch. Suitable starches include, for example: PURITY® 21C (28-1801) sold by National Starch and Personal Care.

The cosmetic composition may comprise, for example, tapioca (also known as tapioca starch). Cosmetic compositions of the present invention may comprise, for example, 0-15% tapioca, and all subranges therebetween; more preferably, 0.001-10% tapioca, and all subranges therebetween; even more preferably, 0.001-5% tapioca, and all subranges therebetween; and most preferably, 0.001-3% tapioca, and all subranges therebetween. Suitable tapiocas include, for example: TAPIOCA PURE (28-1810) sold by National Starch and Personal Care.

The cosmetic composition may comprise, for example, zinc oxide. Cosmetic compositions of the present invention may comprise, for example, 0.05-20% zinc oxide, and all subranges therebetween; 0.1-10% zinc oxide, and all subranges therebetween; more preferably, 0.1-7% zinc oxide, and all subranges therebetween; even more preferably, 0.1-3% zinc oxide, and all subranges therebetween; and most preferably, 0.25-1% zinc oxide, and all subranges therebetween. Suitable zinc oxides include, for example: Kadox 720 sold by Horsehead Corp.

The cosmetic composition may comprise, for example, kaolin, clay, or bentonite. Cosmetic compositions of the present invention may comprise, for example, 0.05-20% kaolin, clay, or bentonite, and all subranges therebetween. More preferably, cosmetic compositions of the present invention may comprise, for example, 0.1-5% kaolin, clay, or bentonite, and all subranges therebetween. Suitable kaolins include, for example: Kaolin 2457BC sold by MPSI.

The cosmetic composition may comprise, for example, additional components, including, for example, titanium dioxide. Other constituents normally found in body powder compositions may also be added to the composition of the present invention, if desired. Such constituents include, but are not limited to, flow agents, medicaments, perfumes, deodorants, disinfectants, antifungal agents, skin protectants, antibacterial agents, anti-caking agents, colorizing agents, stabilizers, antiperspirants, emollients, binders, fillers, extenders, and mixtures and dilutions thereof. Preferably, such additives comprise up to 5%. Nevertheless, depending on factors such as the selection of additive(s) and the intended use of the present invention, greater amounts (up to 25%, for instance) may be used. By way of example, a composition according to the present invention may contain up 25% of an antifungal agent.

Although not wishing to be bound by the following theories, the inventors believe that finely milled corn starch and/or cosmetic talc may produce better adhesion and absorbency and that the starch and/or talc may be used as a pigment carrier; that the sericite cosmetic mica may contribute to an overall soft and silky texture; that the tapioca may act as an aesthetic skin conditioner; and that the iron oxide pigments may provide the coloration and/or tinting effects.

According to one aspect of the present invention, a method of making a cosmetic composition is provided. The starch (e.g., corn starch) and talc (or talc substitute) are dry mixed in no particular order to produce the desired consistency. The pigments (i.e., iron oxide(s)) may break upon pressure to produce a multitude of varying shades. These pigment(s), with other ingredients, are milled with the starch and talc (or talc substitute) according to the desired “base” shade. This desired base shade can be modified by the end user to “fine tune” the final shaded color—according to the user's actual body color. The modification may occur by rubbing or massaging the cosmetic composition into the user's skin.

Generally speaking, most of the cosmetic composition's ingredients are mineral in origin. In accordance with one embodiment of the present invention, the aggregate product comprises talc, mica, zinc oxide, starch, and iron oxide compounds. Depending on the quantity being manufactured, the size of the mixing vessels vary but the method is generally the same regardless of the quantity of finished product. The talc and mica (preferably sericite) compounds are mixed into the mixing vessel first since they generally make up the majority of the product. Next, the zinc oxide and starch compounds are added slowly for a substantially even homogenation. The order in which these components (i.e., the zinc oxide and the starch) are added to the talc/mica mixture is not critical. The iron oxide pigment compounds are then added very slowly in order to adjust to the precise color needed. Adding the proper amount of iron oxide may be the most difficult part of the process, and it may be necessary to take small samples during the addition of the iron oxide in order to test for desired color. The amount of iron oxide depends on various factors, including, but not limited to, desired tint and environmental and/or weather conditions. Individual bottles may be then filled with final product.

In accordance with another embodiment of the present invention, a method of applying a cosmetic composition to a user's skin is provided. The method may comprise the following steps: applying a cosmetic composition to the user's skin and rubbing the cosmetic composition to achieve a desired tint or darkness (e.g., shading). The excess powder, if any, may be removed using various methods, including, for example, gravity, blowing (e.g., generated by breath or fan) or brushing. The method in accordance with this embodiment of the invention may be particularly beneficial to those persons desiring to match non-light skin tones

The product may, for example, be moisture absorbing, moisture inhibiting, and skin soothing while enhancing the beauty of an individual's natural coloring.

Although described herein primarily as a body powder composition, the cosmetic composition of the present invention may be utilized in other cosmetic mediums, such as cream formulations, lipsticks, sun-block products, eye shadow, face powder, hair products, body lotion, self tanners, face bronzers, shimmer products, etc.

In an exemplary embodiment, the powder composition contains the following components:

| Ingredient | Percentage |
|---|---|
| Imperial 200 Cosmetic Talc (Absorbate | 62% |
| and Pigment Carrier) | |
| Sericite Cosmetic Mica (provides silky | 15% |
| texture) | |
| Cosmetic Grade Corn Starch (Pigment | 15% |
| Carrier/Absorbate) | |
| Titanium Dioxide (Sun Protection) | 1.0% |
| Tapioca, Pure (Aesthetic Skin | 1.0% |
| Conditioning and Adherence Agent) | |
| Iron Oxide Pigments | 4.0% |
| Kaolin, Colloidial (Adherence Agent) | 2.0% |

In another exemplary embodiment, the powder composition contains the following components:

| Ingredient | Percentage |
|---|---|
| Talc | 73.5% |
| Sericite Mica | 15% |
| Corn Starch | 5% |
| Brown and Yellow | 4% |
| Iron Oxide Pigments | |
| Tapioca | 0.5% |
| Zinc Oxide | 0.5% |
| Kaolin | 0.5% |

An ordinarily skilled artisan will appreciate that the precise mixture of the types of iron oxide may vary from batch to batch, depending on various factors, including, for example, desired tint as well as weather- and environmental-related considerations. Thus, even if 4% iron oxide is typically used, the precise composition may include more light brown iron oxide in some instance, more brown iron oxide in other instances, and more yellow iron oxide in further still other instances. For example, the 4% iron oxide may consist of 2.75% light brown iron oxide and 1.25% yellow iron oxide. Thus, the precise percentage of iron oxide may not always be determined in advance, and differing ranges of percentages of iron oxide may be appropriate in different circumstances.

The invention is not meant to be limited to these components or the listed proportions. Those of ordinary skill in the art will appreciate that deviations may be made to enhance the composition without affecting product functionality.

Additional usages for the powder may involve different marketing strategies, with the same basic formula, for people of lighter skin tones, so that they may enjoy its application through usage as a sunless tanner. A light-skinned user would apply the product, in the same manner, with the goal of obtaining temporary darker skin (akin to a suntan), possibly without any ill effects relating to sun exposure.

Another use of the product may involve a shimmer product—in substantially the same formulas but with shimmer for cosmetic purposes.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. A method of applying a cosmetic composition to a user's skin comprising the steps of:
applying the cosmetic composition to the user's skin; and
rubbing the cosmetic composition to achieve a desired tint;
wherein the tint varies as a function of the step of rubbing; and
wherein the cosmetic composition comprises:
10-90% talc or talc substitute;
1-50% mica; and
1-10% iron oxide.

2. The method of claim 1, wherein the cosmetic composition further comprises 0-90% starch.

3. The method of claim 1, wherein the cosmetic composition further comprises: 0.001-15% tapioca.

4. The method of claim 1, wherein the cosmetic composition further comprises 0.05-20% zinc oxide.

5. The method of claim 1, wherein the cosmetic composition comprises: 60-80% talc; 10-15% mica; 5-20% starch; and 3-7% iron oxide.

6. The method of claim 5, wherein the cosmetic composition further comprises: 0.001-5% tapioca and 0.1-7% zinc oxide.

7. The method of claim 5, wherein the mica is sericite.

8. The method of claim 5, wherein the starch is corn starch.

9. A method of making a cosmetic composition comprising 50-90% talc, 5-20% mica, 5-20% starch, 0.05-20% zinc oxide, and 1-10% iron oxide, the method comprising the steps of:
mixing the talc and the mica in a vessel;
adding the zinc oxide to the vessel;
adding the starch to the vessel;
adding the iron oxide to the vessel so as to achieve a desired coloration.

10. The method of claim 9, further comprising the step of adding 0.001-10% tapioca to the vessel prior to adding the iron oxide.

11. A cosmetic composition comprising:
10-90% talc or talc substitute;
1-50% mica; and
1-10% iron oxide;
wherein the cosmetic composition has a tint that varies in accordance with the amount the composition is rubbed into a user's skin.

12. The cosmetic composition of claim 11 comprising 50-90% talc.

13. The cosmetic composition of claim 11 comprising 60-80% talc.

14. The cosmetic composition of claim 11 comprising 5-40% mica.

15. The cosmetic composition of claim 11 comprising 10-20% mica.

16. The cosmetic composition of claim 15, wherein the mica is sericite.

17. The cosmetic composition of claim 11 comprising 60-80% talc and 5-20% starch.

18. The cosmetic composition of claim 17, wherein the starch is corn starch.

19. The cosmetic composition of claim 11 comprising 10-90% starch.

20. The cosmetic composition of claim 11 comprising 30-60% starch and 10-30% talc.

21. The cosmetic composition of claim 11 comprising 0.001-15% tapioca.

22. The cosmetic composition of claim 20 comprising 0.001-5% tapioca.

23. The cosmetic composition of claim 11 comprising 0.05-20% zinc oxide.

24. The cosmetic composition of claim 20 comprising 0.1-3% zinc oxide.

25. The cosmetic composition of claim 11 comprising: 60-80% talc; 10-20% mica; 5-20% starch; 3-7% iron oxide; 0.001-5% tapioca; and 0.1-3% zinc oxide.

26. The cosmetic composition of claim 25 consisting essentially of: 60-80% talc; 10-20% mica; 5-20% starch; 3-7% iron oxide; 0.001-5% tapioca; and 0.1-3% zinc oxide.

27. The cosmetic composition of claim 25 further comprising 0.05-20% kaolin.

28. The cosmetic composition of claim 25 further comprising 0.1-5% kaolin.

29. The cosmetic composition of claim 25 further comprising 0.001-10% fragrance.

30. The cosmetic composition of claim 25 further comprising 4-6% fragrance.

31. The cosmetic composition of claim 11 further comprising at least one of the ingredients selected from the group consisting of: kaolin, clay, and bentonite.

32. The cosmetic composition of claim 31, wherein the cosmetic composition comprises 0.1-5% kaolin, clay, or bentonite.

33. The cosmetic composition of claim 11, wherein the cosmetic composition is in the form of a body powder, a cream formulation, a lipstick, a sun-block product, an eye shadow, a face powder, a hair product, or a body lotion, a self tanner, a face bronzers, or a shimmer product.
